(19) United States
(12) Patent Application Publication
(10) Pub. No.: US 2026/0201317 A1
(43) Pub. Date: Jul. 16, 2026

(54) BIODEGRADABLE CARRIER FOR METHANIZATION AND/OR METHANATION REACTION AND METHOD FOR MANUFACTURING

(71) Applicants: UNIVERSITE DE PAU et PAYS DE L’ADOUR, PAU (FR); TotalEnergies OneTech, Courbevoie (FR); Centre National de la Recherche Scientifique, PARIS (FR)

(72) Inventors: Cecilia SAMBUSITI, Pau Cedex (FR); Jean BERNARD, Pau Cedex (FR); Guillaume CAZAUDEHORE, Pau Cedex (FR); Rémy GUYONEAUD, Pau Cedex (FR); Christine PEYRELASSE, Pau Cedex (FR); Florian MONLAU, Pau Cedex (FR); Frédéric LEONARDI, Pau Cedex (FR)

(21) Appl. No.: 19/136,670

(22) PCT Filed: Dec. 9, 2022
(86) PCT No.: PCT/IB2022/000721

§ 371 Date: Jun. 6, 2025

(51) Int. Cl. C12N 1/20; B33Y 10/00; B33Y 80/00; C12M 1/00; C12M 1/107

(52) U.S. Cl. CPC C12N 1/20; B33Y 80/00; C12M 21/04; C12M 23/30; B33Y 10/00

(57) ABSTRACT

The invention relates to a biodegradable carrier (1) suitable for use in a methanization and/or methanation reaction comprising at least one methanization reactor, said biodegradable carrier comprising: —at least 70 % in weight of a biodegradable polymer, —at least one closed cavity (2) preferably formed in the biodegradable polymer, —at least one surface (3) for the growth of microorganisms, the biodegradable carrier is further configured to be in suspension in the at least one methanization reactor. The invention also relates to a method (100) for manufacturing a biodegradable carrier (1) suitable for use in a methanization and/or methanation reaction and a system for methanization and/or methanation reaction comprising at least one methanization reactor and at least one biodegradable carrier (1) suitable for use in a methanization and/or methanation reaction.

### FIELD OF THE INVENTION

The present invention relates to the field of gas and in particular to the methanization and/or methanation reaction.

This invention provides a new biodegradable carrier for methanization and/or methanation reaction and a method for manufacturing.

### DESCRIPTION OF RELATED ART

Currently, global warming is a major issue. To promote the ecological transition, methods such as pyro-gasification, power-to-gas or methanization are processes allowing the production of “green gas”, non-fossil renewable energy, close to carbon neutrality, thus contributing to the transition ecological.

The methanization or the anaerobic digestion (AD) is based on the biological phenomenon of fermentation of organic matter as illustrated in FIG. 1. The anaerobic digestion, in anaerobic conditions (absence of O₂), comprises the transformation of organic matter such as food waste from fruits and vegetables, household waste, agricultural residues (slurry, manure) or even industrial waste such as cereal dust in absence of O₂. This natural degradation occurs thanks to micro-organism. The process of AD may be explained with 4 steps: hydrolysis, acidogenesis, acetogenesis and methanogenesis through acetotrophic, hydrogenotrophic or methylotrophic pathway. These reactions allow the production of two components: biogas and digestate. Indeed, during the anaerobic digestion the organic matter (organic feedstock) is transformed into biogas composed mainly of carbon dioxide (CO₂) and methane (CH₄). This biogas may be purified in order to be injected in natural gas network while eliminating the CO₂.

There are several ways to recover CO₂ such as agricultural uses, growth of micro-algae and other process such as methanation.

Methanation uses CO₂ and preferably CO₂ from biogas with hydrogen to produce CH₄ and water. Two pathways predominate in methanation, namely catalytic methanation and biological methanation. The catalytic methanation consists of transforming hydrogen into methane, in the presence of a catalyst, which triggers the synthesis reaction. This technology is exothermic (which releases heat) and must therefore impose temperature control in the reactor. Biological methanation, a more recent technology, is intrinsically close to the methanization process, of which it is one of the 4 essential reactions. Biological methanation, in-situ or ex-situ, is a technology that uses archaea. It differs from catalytic methanation which uses a catalyst to synthesize H₂ and CO₂ into CH₄. The biological pathway is implemented at a lower temperature and pressure than the catalytic pathway and therefore consumes less energy. On the other hand, the level of development of catalytic methanation is more advanced than biological methanation. In the biological pathway, the CO₂ contained in the biogas placed in the presence of H₂ is then transformed into CH₄ and water, thanks to the action of the archaea contained in the methanization reactor. In this case, it is an in-situ biological methanation. If the methanation process is carried out in another dedicated reactor at the methanization outlet, it is called ex-situ biological methanation. The biological methanation could make it possible to no longer use a biogas purifier at the outlet of the methanization to separate the CO₂ from the CH₄ because it would be converted into CH₄ by injecting hydrogen. Thus, the production of biogas and its enrichment in CH₄ makes it possible to recover CO₂. Indeed, the CO₂ from methanization is partly consumed during methanation to produce methane (biomethane).

Nevertheless, during methanization and/or methanation, the natural degradation occurs thanks to microorganisms. Hydrolytic, acetogenic, fermentative bacteria and methanogenic archaea are the four distinctive microbial community found in a reactor. However, archaea (responsible of the production of methane) tend to be more sensitive to the modification of their environment. Indeed, the addition of CO₂ and/or H₂ can significantly affect the operation of anaerobic digestion and can compromise methanation. The performance of biological methanation is directly linked to gas transfer properties and operating parameters. Indeed, the increase in the partial pressure of H₂ and of dissolved H₂ affects certain oxidation reactions of the volatile fatty acids of the methanization metabolism which can cause biological dysfunctions resulting in a slowing down, or even a blocking of the methane production. These dysfunctions are classically characterized by an increase in long-chain volatile fatty acids.

The challenge of a biological methanation process is to obtain dissolved H₂ levels that do not limit the oxidation of volatile fatty acids, thus allowing the simultaneous production of biogas and the transformation of CO₂ from biogas into CH₄. Furthermore, the biological methanation reaction allows the formation of CH₄ from CO₂ and H₂ thanks to the action of methanogenic archaea present in the anaerobic digestion medium. This reaction therefore requires the solubilization of the gases in the digestion medium. One of the problems of biological methanation lies in the solubilization of these gases and in particular of H₂ which is 24 times less soluble than CO₂ In addition, archaea grow very slowly and often continue conversions during the non-growing phase of their life cycle to gain metabolic energy for maintenance.

In parallel, regarding methanization and/or methanation, the digestate is an important point to consider in order to avoid the toxicity of soil and water. Indeed, the digestate will return for example to the soil by spreading. It is therefore essential that the digestate does not compromise its acceptability for agronomy.

Several technologies of methanization have been developed. For example, the methanization with CSTR for Continuous Stirred Tank Reactor, the methanization with EGSB for Expanded Granular Sludge Bed, the methanization UASB for Up flow Anaerobic Sludge Blanket, the solid state methanization and the fixed bed methanization with carriers. The use of carrier promoting the development of the microorganisms. Cell retention by biofilm formation is a very good way to increase cell density in bioreactors. This requires a large surface solid matrix for the cells to colonize and form a biofilm which contains the metabolizing cells in a matrix of extracellular polymeric substance which the cells secrete.

Different types of carriers exist in the field of waste or pollutants treatment. Carriers may be fixed or mobile. For example, in moving bed biofilm reactor designed for water treatment, several type of carriers such as Anoxkaldnes® are used and allow the growth of micro-organism in wastewater treatment. These carriers move through the water to be purified, to bring the greatest number of bacteria into contact with the greatest quantity of pollutant. However, it is necessary to provide continuous movement for the carriers to move, and to use several carriers to ensure an adequate load of microorganisms. In addition, these carriers are not suitable for the production of biogas and in particular for methanization and/or methanation reactors. In addition, they are not compatible with the recovery of biogas and its enrichment in biomethane. Moreover, these carriers are not biodegradable and strongly compromise the acceptability of the digestate when it returns to the ground. This type of carrier leaves an imprint at the bottom of the reactor. Furthermore, this carrier (Anoxkaldnes®) does not make it possible to ensure the control of the development of microorganisms which leads to the clogging of the carriers then impacting the environment of the micro-organisms and therefore their efficiency and performance.

The document EP0575314 discloses a carrier for water purification. This carrier presents a density of between 0,90 and 1,20 in order to be kept in the medium of a reactor with mixing means. Such carrier may have certain curvatures to ensure less contact with another carrier or with walls of reactor ensuring a better growth of micro-organisms. However, this carrier requires agitation of the medium to be in contact with organic matter and must be pumped out after use or even evacuated after use. In addition, this carrier is not suitable for the production of biogas and even less to the production of biomethane. Moreover, this carrier ends up at the bottom of the reactor, which is not acceptable in the field of methanization and/or methanation.

Furthermore, fixed or mobile carriers have high treatment yields, fixed carriers are frequently obstructed or clogged by biological deposits requiring cleaning or replacement operations, mobile carriers due to a reduction in the specific surface area and are subject to the deterioration and have a limited lifespan.

Simultaneously, carriers are currently made of plastic. Faisal et al. (2020-Biomethane enhancement via plastic carriers in anaerobic co-digestion of agricultural wastes, Biomass Conversion and Biorefinery, volume 12, pages 2553-2565) demonstrated that the addition of bacterial biofilm carriers from fossil plastics (i.e HDPE) significantly improved methane production and microscopic analysis highlighted the development of bacterial biofilms on the surface. In parallel, in recent years, there has been a growing interest in the addition of conductive materials in biological processes in order to improve stability and performance as illustrated in FIG. 2. However, such solutions are expensive, made from fossil resources and can contaminate the digestate.

The document EP3240901 discloses a carrier made of pieces of polyethylene. The biofilm growths after two weeks, and the thickness of the biofilm does not change. This results in a continuous detachment and renewal of the colonization. As explained, this type of carrier does not degrade during digestion and remains in the digestate.

The document EP3555258 discloses a membrane and preferably a hydrophobic microporous membrane, between a gaseous and a liquid phase also allowing the growth of a biofilm. The biofilm occupies the pores of the membrane or a part of the pores. The membrane may be tubular, capillary, hollow fiber, flat sheet and made from polypropylene (PP), polyethylene (PE), polyvinylidene fluoride (PVDF) and polyvinyl chloride (PVC) or other dense polymer such as polysulphone, polyimide or cellulose. In addition, several membranes may be arranged in one or more common networks. Furthermore, said membrane used in this document is used in ex-situ methanation reactors. This type of reactor has very different characteristics from an in-situ methanation reactor. Thus, it is not possible to adapt this type of membrane to in-situ methanation. Again, this type of carrier called membrane in this document, does not degrade during digestion, and remains in the digestate.

Thus, most reactors processing agricultural and/or agro-industrial waste need good biogas yield. Indeed, it is particularly advantageous if the methanization and/or the methanation reaction will be able to produce more biogas and/or more CH₄ in order to reduce the CO₂. It is therefore important to be able to optimize and maximize the production of biogas and its enrichment in biomethane without inhibiting anaerobic digestion. As explained, it is essential to ensure the biologic stability and the resilience of the process in relation to disturbances and in particular the introduction of H₂.

There is therefore a need for new means and methods capable of transforming organic matter into biogas and ensuring the recovery of CO₂ and preferably the production of biomethane by methanization and/or methanation and preferably by biological in-situ methanation while guaranteeing biological stability and the production of plastic-free digestate.

### SUMMARY OF THE INVENTION

The following sets forth a simplified summary of selected aspects, embodiments and examples of the present invention for the purpose of providing a basic understanding of the invention. However, the summary does not constitute an extensive overview of all the aspects, embodiments and examples of the invention. The sole purpose of the summary is to present selected aspects, embodiments and examples of the invention in a concise form as an introduction to the more detailed description of the aspects, embodiments and examples of the invention that follow the summary.

The invention aims to overcome the disadvantages of the prior art. In particular, the invention proposes a biodegradable carrier suitable for use in a methanization and/or methanation reaction comprising at least one methanization reactor, said biodegradable carrier comprising:
at least 70 % in weight of a biodegradable polymer,
at least one closed cavity preferably formed in the biodegradable polymer,
at least one surface for the growth of microorganisms,
   the biodegradable carrier is further configured to be in suspension in the at least one methanization reactor. of biogas through methanization (or anaerobic digestion) and biomethane content in biogas during the in-situ biological methanation reaction while ensuring the production of a digestate free of plastic.

Indeed, the carrier according to the invention promotes the development of micro-organisms and preferably of archaea. Indeed, thanks to the carrier in suspension in the at least one methanization digester, the anaerobic condition, nutrition condition, temperature and other technical parameters are favored for their development. In addition, biodegradable polymer also allows to participate in their development and in particular for hydrogenotrophic archaea in combination with oxidizing bacteria. Advantageously, the carrier makes it possible to limit and reduce the supply of dihydrogen (H₂) into the reactor and in particular to remain within the stoichiometry of the methanation reaction in order to improve the enrichment in biomethane.

The presence of biodegradable polymer ensures the acceptability of the digestate.

A carrier according to the invention allows to control the reaction and in particular the stability and the performances of the in-situ biological methanation while promoting the development of micro-organisms. The carrier allows to minimize or even avoid the addition of H₂ to the reactor.

The invention allows to maximize the production of biogas and in particular biomethane without inhibiting anaerobic digestion.

According to other optional features of the biodegradable carrier according to the invention, it can optionally include one or more of the following characteristics alone or in combination:
biodegradable carrier is configured to be both floating and immersed in the at least one methanization reactor, preferably the biodegradable carrier is maintained in suspension in the at least one methanization reactor by stirring allowing to the carrier to be in contact with the medium and to stay in anaerobic condition
the methanization reactor is a biological in-situ methanation reactor, preferably the methanization reactor allows at the same time a methanization and an in-situ methanation reaction, the same reactor allows to reduce the cost and to facilitate the enrichment of biogas in biomethane
the biodegradable polymer is a design manufacturing from a polymeric composite composition, allowing to response to the need of design and to improve the adherence of micro-organism on the carrier
the biodegradable polymer comprises natural polymers, polysaccharides and/or proteins, polymers synthesized by bacteria, polyhydroxyalkanoate (PHA) such as polyhydroxybutyrate (PHB) and/or polyhydroxyvalerate (PHV) and/or polyhydroxyhexanoate (PHH); and/or synthetic polymers derived from biotechnology of natural monomers, such as polylactic acid (PLA) and/or other aliphatic and/or aromatic polyesters and/or copolyesters, and/or mixture thereof., allowing to the digestate to be free of plastic and ensuring the acceptability of digestate while ensuring optimal development of micro-organisms
the biodegradable polymer forms the growth surface for micro-organisms favouring the space for the growth and the development of micro-organisms
the biodegradable carrier presents a density between 0.70 and 1.00 ensuring the suspension of the carrier in the reactor and avoid its sedimentation in the reactor,
the biodegradable carrier presents a conductivity between 10⁻² and 10² S/cm allowing to improve the DIET,
the polymeric composite composition comprises at least one additive, preferably for improving the properties of the carrier.
the polymeric composite composition comprises at least one additive selected from: nanotube, biochar, carbon black, activated charcoal, glycerol, fiber, mineral additive and/or biological additive and/or mixture thereof.
the polymeric composite composition comprises an additive content between 10% et 30% in weight, preferably in the total weight of the polymeric composite composition.
the at least one closed cavity forms a total hollow volume of at least 10% of the volume of the biodegradable carrier ensuring the suspension of the carrier
the biodegradable carrier presents at least one indent and/or at least one hole and/or at least one segment, allowing to improve the surface available for the growth of micro-organism and preventing the formation of clumps or clogs,
the biodegradable carrier has a biodegradable time between 60 and 120 days which ensures both enough time for the growth of micro-organisms and the necessary reactions while being degradable.
the biodegradable carrier has a ratio surface-volume between 200 and 1000 m²/m³, preferably between 500 and 1000 m²/m³, ensuring a biofilm and carrier ratio adapted both for growth and development and for the available surface.
the biodegradable carrier has a roughness wherein the Ra is between 100 and 200 μm, which favoring the adherence of micro-organisms

According to another aspect, the invention can also relate to a method for manufacturing a biodegradable carrier suitable for use in a methanization and/or methanation reaction comprising at least one methanization reactor, said method comprising:
A step of providing a polymeric composite composition,
A step of design manufacturing

According to other optional features of method according to the invention, it can optionally include one or more of the following characteristics alone or in combination:
a step of extruding,
the step of design manufacturing is selected between 3D printing such as stereolithography, laser sintering, multi-jet printing, modeling by fused deposition modelling, and/or injection.

According to another aspect, the present invention can also relate to a system for methanization and/or methanation reaction comprising at least one methanization reactor and at least one biodegradable carrier suitable for use in a methanization and/or methanation reaction comprising at least one methanization reactor according to the invention.

### BRIEF DESCRIPTION OF THE DRAWINGS

The foregoing and other objects, features and advantages of the present invention will become more apparent from the following detailed description when taken in conjunction with the accompanying drawings in which:

FIG. 1 is a schematic view of the process of a classic methanization according to the prior art.

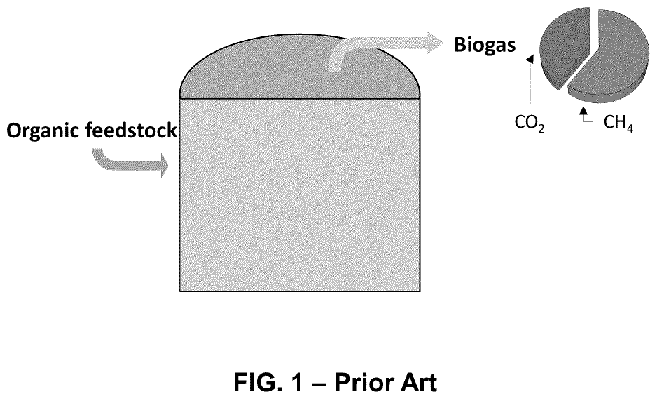

FIG. 2 is a schematic view of the process of a classical methanization with an electrical conducting carrier.

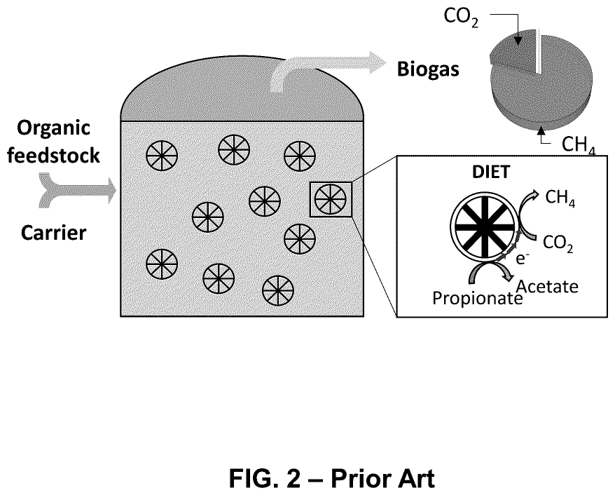

FIG. 3 is a schematic view of a biodegradable carrier according to an embodiment of the present invention.

FIG. 4 is a schematic sectional view of a biodegradable carrier according to one embodiment of the invention.

FIG. 5 is a schematic view of a biodegradable carrier according to an embodiment of the present invention. The FIG. 5A is a front view. The FIG. 5B is a top view.

FIG. 6 is a schematic sectional view of a support according to one embodiment of the invention.

FIG. 7 is a flowchart of a method according to an embodiment of the present invention.

FIG. 8 is a schematic view of a system according to an embodiment of the invention.

Several aspects of the present invention are disclosed with reference to flow diagrams and/or block diagrams of methods, devices according to embodiments of the invention.

On the figures, the flow diagrams and/or block diagrams show the architecture, the functionality and possible implementation of devices or systems or methods, according to several embodiments of the invention.

In some implementations, the functions associated with the box may appear in a different order than indicated in the drawings.

For example, two boxes successively shown, may be executed substantially simultaneously, or boxes may sometimes be executed in the reverse order, depending on the functionality involved.

Each box of flow diagrams or block diagrams and combinations of boxes in flow diagrams or block diagrams may be implemented by special systems that perform the specified functions or actions or perform combinations of special equipment.

### DETAILED DESCRIPTION

A description of example embodiments of the invention follows.

In the following description, by “polymer” is meant either a copolymer or a homopolymer. The term “copolymer” means a polymer grouping together several different monomer units and the term “homopolymer” means a polymer grouping identical monomer units. By “block copolymer” is meant a polymer comprising one or more uninterrupted blocks of each of the distinct polymer species, the polymer blocks being chemically different from each other and being linked together by a covalent bond. These polymer blocks are also called polymer blocks.

The term “polymeric composition” may refer to a material which contain as an essential ingredient a polymer and which, at some stage in its processing into finished products, can be shaped by flow. The “polymer composition” includes polymers but can also include other compounds or materials. Thus, the “polymeric composition” refers to all types of compounds, polymers, oligomers, monomers, copolymers or block copolymers.

The term “polymeric composite” is understood to mean, within the meaning of the invention a multicomponent material comprising at least two immiscible components in which at least one component may be a polymer and the other component can for example be an additive.

The term “biodegradable” may refer to a polymer or a composite capable of meeting the criteria and requirements set out in the EN 13432:2002 standard. Preferably, in the meaning of the invention, “biodegradable” may refer to a polymer being converted to 90 % or higher to carbon dioxide and/or methane in 6 months of industrial composting. The degradation can also occur in methanization, home composting or soil incubation or in a combination thereof. The compost or digestate resulting from the plastic degradation should not be phytotoxic. The polymer and composite should have limited concentration of different metals.

The term “carrier” may refer to a physical mean adapted to be colonized by micro-organisms.

The term “suspension” may refer to at least one carrier in a liquid medium said at least one carrier before the colonization is preferably maintained in the liquid without sinking and/or touching the bottom and without exceeding the liquid/air interface or the meniscus formed by the liquid medium at the interface.

The term “immerge” may refer to a carrier which is suspension in the medium without exceeding the liquid/air interface.

The term “cavity” may designate a preferably closed empty space, without limitation of shape. Cavity can refer to air trapped in an enclosed space. This space can be physically delimited by the biodegradable polymer by at least one wall and/or in the polymeric composite composition itself.

The expression “growth surface” may refer to the entire outer surface of the biodegradable carrier. Preferably the growth surface is the surface in contact with the medium in the digester.

As mentioned, the methanization is an approach to produce biogas. During anaerobic digestion microorganisms break down biomass into methane and carbon dioxide. The process of anaerobic digestion usually begins with the bacterial hydrolysis of input materials. These materials are broken down into derivatives which become available to other micro-organisms and other reaction as disclosed above. The biogas from methanization can be used directly or converted into other forms of renewable energy or used in the CO₂ recovery. In particular in in-situ biological methanation which allows to transform CO₂ from biogas in biomethane and which consumes less energy than catalytic methanation and does not require the addition of an additional reactor compared to ex-situ biological methanation or additional CO₂ supply. Consequently, in order to maximize the recovery of CO₂, the production of biogas and its enrichment in biomethane (production of biomethane) need to be increase.

However, as explained, the ecosystem of the microbial medium can be very fragile and sensitive to changes in raw material, the surrounding bacterial environment or even gas environment as H₂. In addition, the growth and reproduction of microorganisms and in particular of methanogenic microorganisms is particularly slow.

The carriers developed to increase the production of biogas are limited in methanization reactors and even more in biological in-situ methanation. In addition, these carriers can contaminate the digestate which can sediment in the reactors or even can flow into the reactors either as soon as they are introduced into the reactor or as the microorganisms develop, thereby contaminating the digestate and disturbing the ecosystem of the terrestrial and aquatic ecosystem after spreading.

There is therefore a need during the methanization and/or methanation reaction for new means and methods making it possible to ensure the maximization of biogas and its enrichment in biomethane while favoring the development of microorganisms and ensuring the control of their development and preserving the digestate from any contamination or toxicity.

According to a first aspect, the invention relates to a biodegradable carrier 1 suitable for its use in a methanization reactor. Preferably, the invention relates to a biodegradable carrier adapted for a methanization reactor, and more preferably a biodegradable carrier for a methanization reactor. Advantageously, the methanization reactor is suitable for a biological in-situ methanation. A methanization reactor according to the invention may correspond to a biological in-situ methanation reactor. In addition, a biodegradable carrier according to the invention is preferably intended for a methanization and/or methanation reaction.

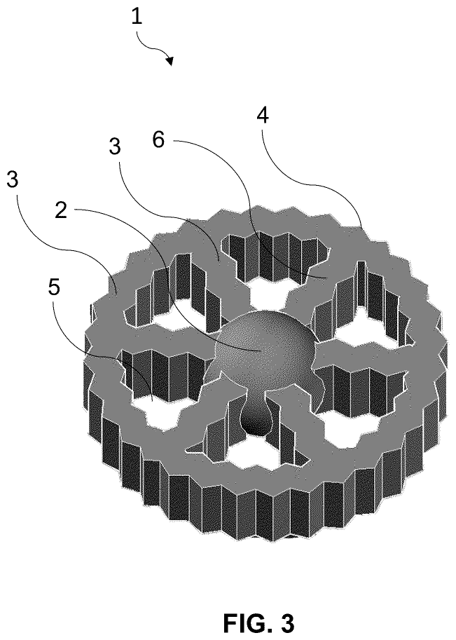

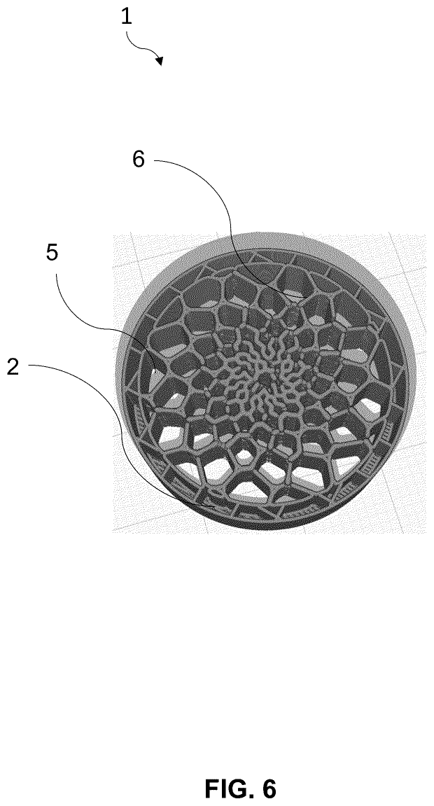

In particular, as illustrated in FIGS. 3 to 6, a biodegradable carrier 1 comprises:
at least 70% in weight of a biodegradable polymer,
at least one closed cavity 2 preferably formed in the biodegradable polymer,
at least one surface 3 for the growth of microorganisms.

A biodegradable carrier 1 according to the invention is further configured to be in suspension in the methanization reactor.

A biodegradable carrier comprises at least 70% in weight of a biodegradable polymer. Preferably, a biodegradable carrier comprises at least 75% of a biodegradable polymer, more preferably at least 80% in weight of a biodegradable. A biodegradable carrier may comprise at most 90% in weight of a biodegradable polymer, preferably at most 88% in weight of a biodegradable polymer, more preferably at most 85% in weight of a biodegradable polymer. A biodegradable carrier may comprise between of 70% and 90% in weight of a biodegradable polymer, preferably between of 75% and 88% in weight a biodegradable polymer, more preferably between of 80% and 85% in weight of a biodegradable polymer.

A biodegradable polymer may be a design manufacturing from a polymeric composite composition. A polymeric composite composition comprises preferably polymer, and/or oligomer, and/or monomer and/or a precursor and another component such as an additive.

A design manufacturing may be selected between 3D printing, such as stereolithography, laser sintering, multi-jet printing, modeling by deposition of fused deposition modeling, and/or injection. Preferably, a design manufacturing according to an embodiment of the present invention is selected between an extrusion, a 3D printing and/or an injection. More preferably, a design manufacturing comprises an extrusion and a 3D printing. A design manufacturing allows to improve the roughness of a biodegradable carrier. A design manufacturing allows to vary design and to adapt the quantities to be produced according to the need. The roughness improves the growth surface of micro-organisms and the adhesion and consequently, to start the methanization and/or the methanation quickly because the micro-organisms adhere faster and with a stronger adhesion compared to a smooth or less rough support. Preferably, the roughness may be quantifying according to the parameter Ra. Advantageously, the biodegradable carrier, preferably the growth surface of microorganism, may have a roughness wherein the Ra may be between 100 and 200 μm, preferably between 150 and 200 μm.

In addition, a design manufacturing and preferably an additive manufacturing, allows to increase the freedom of design. For example, the 3D printing makes it possible to increase the roughness, to put air in the carrier but also to be able to make holes in the carrier.

Back to the polymeric composite composition, the polymer of the polymeric composite composition and more preferably the polymer of the biodegradable polymer can come from renewable carbon sources (e.g., agricultural cultivation, biomass conversion). However, it can be also derived from petrochemical carbon sources. Biodegradable polymers within the meaning of the present invention may correspond to natural polymers, polysaccharides (for example starch, cellulose, chitin, chitosan, alginate) and/or proteins (animal or vegetable); to polymers synthesized by bacteria (fermentation), such as the polyhydroxyalkanoate (PHA) and/or polyhydroxybutyrate (PHB) and/or polyhydroxyvalerates (PHV); and/or polyhydroxyhexanoate (PHH) and/or to synthetic polymers derived from biotechnology of natural monomers, such as polylactic acid (PLA) and/or other aliphatic and/or aromatic polyesters and/or copolyesters and/or mixture thereof.

More preferably the polymer of the polymeric composite composition and even more preferably the biodegradable polymer is selected from all types of biodegradable EN 13 432:2002 compounds, polymers, oligomers, monomers, copolymers. However, it would not be departing from the scope of the invention if the polymeric composite composition or the biodegradable polymer comprises up to 10% by weight, preferably less than 5% by weight of another non-biodegradable polymer. In a preferred but non-limiting embodiment, the polymer of the polymeric composite composition and preferably the biodegradable polymer is selected from starch, PLA; PHA (e.g., PHB, PHBV, PHBH, PHH), PCL, PBAT, PBS. and mixtures thereof.

More preferably, the polymeric composite composition and even more preferably the biodegradable polymer may comprise starch and/or PHA (e.g., PHB, PHBV, PHBH) According to an embodiment of the present invention, the biodegradable carrier and preferably the polymeric composite composition may comprise at least one additive. Preferably, the biodegradable polymer is bonded to an additive. A bond may be a chemical bond or a physic bond such as encapsulation.

The polymeric composite composition may comprise at least one additive selected from: carbon nanotube, biochar, carbon black, activated charcoal, glycerol, fiber, mineral additive and/or biological additive and/or mixture thereof. Preferably, the polymeric composite composition comprises an additive which can be design manufacturing with the biodegradable polymer.

A fibrous reinforcement (fiber additive) may comprise an assembly of one or more fibers, generally several fibers, said assembly being able to have different forms and dimensions; one-dimensional, two-dimensional, or three-dimensional. The one-dimensional form corresponds to linear long fibers. The two-dimensional form corresponds to nonwoven reinforcers or fibrous mats or woven rovings or bundles of fibers, which may also be braided. The three-dimensional form corresponds, for example, to stacked or folded nonwoven fibrous reinforcers or fibrous mats or stacked or folded bundles of fibers or mixtures thereof; an assembly of the two-dimensional form in the third dimension. The fibers may be discontinuous or continuous. When the fibers are continuous, the assembly thereof forms fabrics.

The origins of the fibers constituting the fibrous reinforcer may be natural. Natural fiber may be plant fibers and/or wood fibers, animal fibers or mineral fibers. Plant and/or wood fibers are, for example, lignocellulosic fibers, such as sisal, jute, hemp, linen, cotton, coconut, and banana fibers. Animal fibers are, for example, wool or fur fibers.

A mineral reinforcement (mineral additive) may be basalt fibers, carbon fibers, boron fibers or silica, glass, talc and/or calcium carbonate.

A biological additive may be enzymes such as lipase, esterase, and/or mixture thereof.

Advantageously an additive allows to improve some characteristics of the biodegradable carrier.

Indeed, some additives such as biochar or activated charcoal or carbon black allow to improve the conductivity of the biodegradable carrier. Consequently, the DIET (Direct Interspecies Electron Transfer) is facilitated. DIET allows to improve the methane production yield by reducing carbon dioxide to methane.

The biodegradable carrier may have a conductivity between 10⁻² and 10² S/cm, preferably between 10⁻¹ and 10² S/cm and more preferably between 10 and 10² S/cm. The conductivity may be measured by an ohmmeter or an electrometer using the two-wire or four-wire method.

Other additives such as glycerol allow to improve the homogeneity of the biodegradable carrier.

Other additives such as fiber and mineral allow to improve the reinforcement of the biodegradable carrier.

Other additives such as enzymes allow to improve the biodegradability of the biodegradable carrier.

According to the embodiment wherein the biodegradable carrier and preferably the polymeric composite composition may comprise at least one additive, the polymeric composite composition may comprise an additive content between of 10% and 30% in weight, preferably between 10% and 20% and even more preferably less than or equal to 10% in weight, preferably in weight of the polymeric composite composition and more than or equal to 0% in weight. Said rate allows to improve the characteristics of the biodegradable carrier and in particular the conductivity and consequently the DIET, improving the biomethane content in biogas without disturbing the growth of microorganisms. In addition, the biodegradable polymer and/or the polymer composite composition allows to improve the hydrogenotrophic and acetotrophic microorganisms.

A biodegradable carrier comprises a biodegradable polymer which provides both a substrate for micro-organisms but also a growth surface. Indeed, the biodegradable carrier may be colonized by micro-organisms. In addition, and in a particularly advantageous way, a biodegradable polymer or a polymeric composite composition makes it possible to avoid contamination of the digestate by non-biodegradable plastic.

The biodegradable carrier comprises at least one surface 3 for the growth of microorganisms. Preferably, the biodegradable polymer forms the growth surface for microorganisms. The growth surface should be sufficient to ensure sufficient space for the development of microorganisms and their growth (colonization).

The growth surface may have different form and/or dimension as shown in FIGS. 3 to 6. Preferably the growth surface is a volume, or a growth surface is in three dimensions.

The growth surface may be delimited by at least one an outer wall or by a double outer wall.

The biodegradable carrier may have at least one segment 6, at least one indent 4, and/or at least one hole 5, preferably the growth surface may have at least one segment and/or at least one indent. At least one segment and/or at least one hole and/or at least one indent allows to maximize the ratio between the growth surface and the volume of the biodegradable carrier. In addition, the at least one segment and/or at least one hole and/or at least one indent allows to create protected spaces in the carrier for the development and growth of micro-organisms.

Preferably the growth surface may be rough. The roughness may be between 100 and 200 μm for the Ra.

According to one embodiment of the invention, the biodegradable carrier comprises at least one indent 4. Preferably the growth surface may comprise at least one indent. An indent makes it possible to increase the available growth surface in comparison with the same carrier without indent, and without increasing the dimensions of the biodegradable carrier.

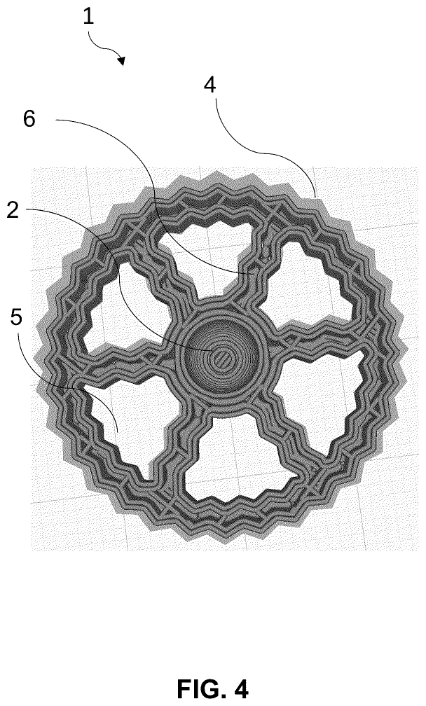

For example, an indent as exposed in the FIGS. 3 to 4 may have any form, shape and/or dimension.

In a particular embodiment, the biodegradable carrier may comprise between 1 and 100 indents. The indent may be arranged in the growth surface and/or in the at least one segment.

According to an embodiment with more than at least one indent, said indents may have different dimensions and/or shape from each other.

According to a particular embodiment, the biodegradable carrier may have at least one segment. The biodegradable carrier may have at most 100 segments. Preferably each segment may be separated by at least one hole. A segment 6 may have at least one indent 4. A segment may have any shape and/or dimension.

According to an embodiment with more than at least one segment, said segment may have different dimensions and/or shape from each other.

The biodegradable carrier can also comprise at least one hole 5. At least one hole makes it possible to avoid the occlusion of the carrier by microorganisms and contributes to their proper development. The biodegradable carrier can have at most 100 holes. The at least one hole may be arranged on the surface of growth and/or the at least one segment.

The at least one hole may have different dimensions and/or shape.

According to an embodiment with more than at least one hole, said hole may have different dimensions and/or shape from each other The biodegradable carrier may be delimited by at least one outer wall or a double outer wall. In addition, the biodegradable which is preferably in 3 dimensions may have a fill rate between 10% and 60% preferably with said polymeric composite composition.

The biodegradable carrier may have a surface/volume ratio between of 200 and 1000 m²/m³, preferably between 500 and 1000 m²/m³. The surface represents the space available to accommodate a biofilm, the volume is the place it occupies in the reactor. If the ratio is low, there is relatively little biofilm compared to the space that the carrier takes up in the reactor. There is therefore a limited effect of the carrier. In comparison, a ratio too high may be a risk of having very small holes or pores which will clog quickly. Thus, there will only be a small surface which will be available for microbial colonization. A ratio between 200 and 1000 m²/m³ allows to ensure a biofilm and carrier ratio adapted both for growth and development and for the available surface.

The biodegradable carrier may have different form and/or dimension, as illustrated in FIG. 3 to 6.

For example, the biodegradable carrier may comprise a length between 10 mm and 35 mm, preferably between 15 mm and 30 mm.

The biodegradable carrier can for example comprise a width between 1,0 mm and 35,0 mm, preferably between 2,0 mm and 30 mm.

The biodegradable carrier can for example comprise a thickness between 3 mm and 13 mm preferably between 6 mm and 10 mm.

The biodegradable carrier can comprise an external diameter between 20 mm and 35 mm, preferably between 25 mm and 30 mm.

The biodegradable carrier can comprise an internal diameter between 19,0 mm and 29,9 mm, preferably between 20,0 mm and 25,0 mm.

The biodegradable carrier can have a polygonal, circular, tubular shape. Preferably the biodegradable carrier can have a circular shape. By circular is meant any shape reminiscent of a circle.

Advantageously, the biodegradable carrier may have a biodegradable time between 60 and 120 days, preferably between 65 and 115 days and more preferably between 70 and 110 days. The biodegradable time of the biodegradable carrier depends on the development of the microorganisms. Indeed, during their development the biodegradable carrier is consumed. In addition, such a biodegradable time makes it possible to ensure sufficient time for the development of the microorganisms and the progress of the chemical reactions in the reactors favoring the production of biogas and consequently its enrichment in biomethane. Thus, this makes it possible to ensure both the development of microorganisms, the improvement of the production of biogas and enrichment in biomethane and to avoid contamination of the digestate.

It should be noted that more the microorganisms will grow on the biodegradable carrier, the heavier the biodegradable carrier will be and will sink in the methanization reactor. In addition, this increases the risk of sedimentation in the methanization reactor. However, as disclosed above, the biodegradable carrier may have a biodegradable time of between 60 and 120 days to allow time for the microorganisms to grow and for the chemical reactions to take place.

According to one embodiment of the invention, the biodegradable carrier comprises at least one closed cavity 2. Such a closed cavity reduces the surface available for the development of microorganisms. However, such a closed cavity makes it possible to maintain the biodegradable carrier in suspension in the methanization reactor, which makes it possible to promote the environment for the development of microorganisms. Thus, the biodegradable carrier is configured to be in suspension in the methanization reactor. By suspension is meant the fact that the carrier is maintained in the methanization reactor without flowing and without floating above the liquid/gas interface. Consequently, the biodegradable carrier is configured to be both floating and immersed in the methanization reactor. This makes it possible to provide microorganisms with an environment conducive to their development and to avoid any risk of sedimentation and contamination of the digestate.

The at least one closed cavity may have a length between 3 mm and 15 mm, preferably between 5 mm and 10 mm. Preferably the dimension of the closed cavity is function of the desired density, and preferably between 0.7 and 1.00. Indeed, thanks to a controlled density, the biodegradable carrier can float while being immersed before colonization and remains in suspension during the development of microorganisms.

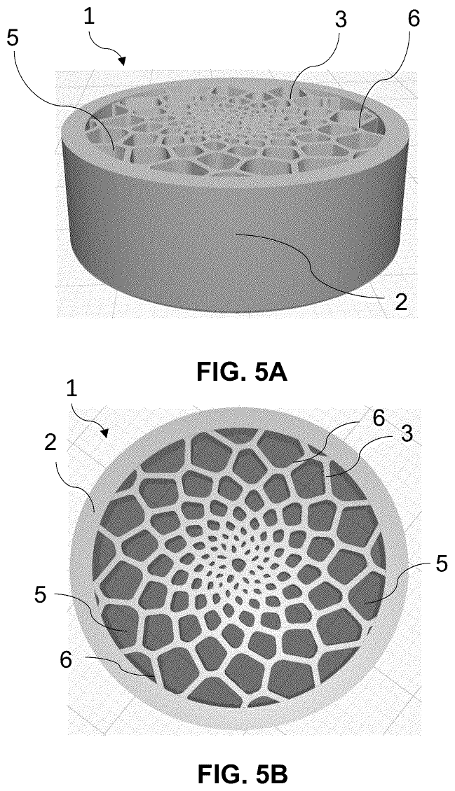

The closed cavity may be arranged anywhere in the biodegradable carrier. For example, the closed cavity may be arranged in center of the biodegradable carrier as exposed in the FIG. 3, and/or in the periphery of the biodegradable carrier as exposed in the FIG. 5, and/or in the at least one indent, in the at least one segment and/or in the biodegradable polymer and/or in the polymeric composite composition.

The biodegradable carrier may have between 1 and millions of closed cavities. For example, the closed cavity may correspond to the air trapped inside the biodegradable carrier, inside the polymeric composite composition and/or in the at least one indent and/or in the at least one segment and/or in the at least one outer wall and/or according to the filling rate of the biodegradable carrier. Preferably the closed cavity is filled with air.

Preferably, the at least one closed cavity may form a total hollow volume of at least 5% of the volume of the biodegradable carrier, preferably at least 7% and more preferably at least 10% of the volume of the biodegradable carrier. The at least one closed cavity may form a total hollow volume of at most 50% of the volume of the biodegradable carrier, preferably at most 40% and more preferably at least 30%. The at least one closed cavity may form a total hollow volume between 5% and 50% of the volume of the biodegradable carrier, preferably between 7% and 40% and more preferably between 10% and 30%.

According to another example, the biodegradable carrier may have several closed cavities, each of them can be arranged differently in the biodegradable carrier and/or in the polymeric composite composition, of different shape and/or size.

The closed cavity may be concave or convex. The closed cavity may be of any shape. The at least one closed cavity may have different form and/or dimension. According to an embodiment closed cavities may have different form and/or dimension from each other. For example, the at least one closed cavity may be circular, tubular, polygonal.

The at least one close cavity allows to keep the biodegradable carrier under the meniscus (under the liquid/air interface) while keeping the biodegradable carrier immersed in the medium without it sinking. Thus, even with the development of micro-organisms and/or the biodegradable time of the biodegradable carrier, the biodegradable carrier remains submerged without sinking and therefore without sediment out. The biodegradable carrier does not contaminate the digestate.

A biodegradable carrier according to the invention can have a density of between 0.70 and 1.00, preferably between 0.80 and 0,99, more preferably between 0.85 and 0.95, even more preferably between 0.90 and 0.95. Density can be measured according to the mass and the volume of the biodegradable carrier. Such a density makes it possible to ensure the suspension of the biodegradable carrier, while considering the density of the biodegradable carrier and the growth of the microorganisms. In addition, additives such as the fibers described above and/or the number of closed cavities can also make it possible to modulate the density to ensure the suspension.

In a preferred but non-limiting embodiment of the invention, the at least one closed cavity may correspond to a sphere as exposed in FIG. 3 or a circular periphery wall as exposed in FIG. 5. Preferably, the at least one closed cavity is arranged in the biodegradable carrier according to the filling rate. Indeed, such a shape makes it possible to reduce the space not available to microorganisms without causing an increase in the dimensions of the biodegradable carrier so that it remains suitable for a methanization reactor.

The biodegradable carrier is suitable for use in a methanization reactor, and preferably is intended for methanization reactor. Indeed, such biodegradable carrier allows to improve the production of biogas during the methanization reaction while ensuring the production of a non-toxic digestate and the growth of microorganisms. The biodegradable carrier is also suitable for use in methanation reactor, preferably intended for a biological methanation reactor, and more preferably for a biological in-situ methanation reactor. According to an embodiment of this invention, the methanization reactor is a biologic in-situ methanation reactor. Preferably, the biodegradable carrier is suitable for use in methanization and preferably intended for biological in-situ methanation.

The methanization and the methanation present several advantages such as a double recovery of organic matter and energy; a reduction in the quantity of organic waste to be treated by other channels; a reduction in greenhouse gas emissions, possible treatment of greasy or very wet organic waste, limitation of odor emissions.

A biodegradable carrier according to the invention also makes possible to promote the syntrophic relation based on DIET between oxidizing bacteria and methanogenic archaea.

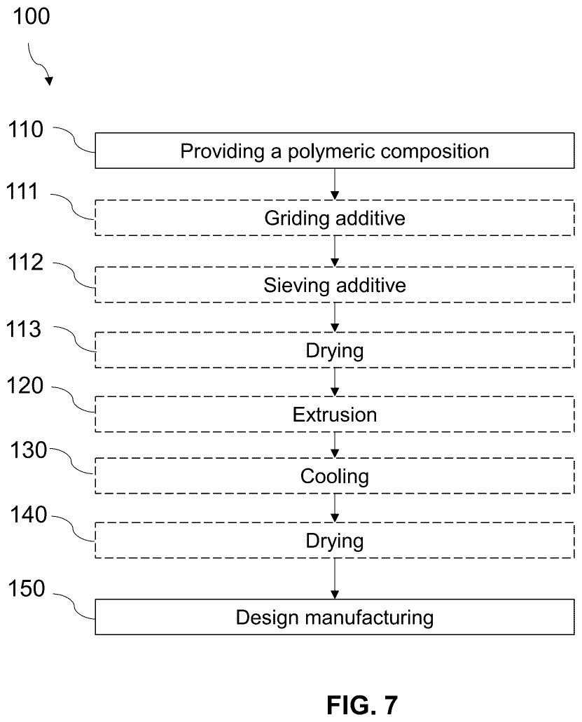

According to another embodiment, the invention relates to a method 100 for manufacturing a biodegradable carrier suitable for use in a methanization, preferably for methanization and/or methanation reaction. More preferably, the method may be a method for manufacturing a biodegradable carrier according to the invention. Such method may be illustrated with the FIG. 7. More preferably the biodegradable carrier may be the biodegradable carrier as disclosed above.

The method 100 for manufacturing a biodegradable carrier may comprise a step of providing 110 a polymeric composite composition and a step of design manufacturing 150.

The step of providing a polymeric composite composition may comprise an optional step of grinding 111 the at least one additive. The step of grinding may be implemented by a grinder. The step of grinding allows to provide a specific granulometry for the design manufacturing.

The step of providing a polymeric composite composition may comprise a step of sieving 112 the at least one additive. The step of sieving may be implemented by a sieve with a predetermined dimension. The step of sieving allows to select a specific granulometry for the design manufacturing.

Preferably the granulometry may be comprised between 1 and 100 μm.

The step of providing a polymeric composite composition may comprise a step of drying 113 the at least one additive and/or the biodegradable polymer. Preferably the step of drying may be implemented by a heater, preferably at a temperature between 65 and 105° C. The step of drying allows to eliminate the humidity.

The step of providing a polymeric composite composition may comprise a step of extruding 120. The step of extruding may be implemented by a twin-screw extruder or a single-screw extruder. This step of extruding allows the mixing and/or the shaping of the polymeric composite composition, comprising the at least one additive or not, which consists of pushing the polymeric composite composition to become fluid through an extrusion die. The step of extruding may comprise a step of determining machine parameter. For example, the step of determining machine parameter may comprise the determination of the temperature, the rotational speed. Preferably, the machine parameters are determined based on the polymeric composite composition. According to an embodiment the temperature may be between 150 and 200° C. According to an embodiment, the rotational speed may be between 50 and 150 rpm.

The step of extruding may comprise a step of introduction the polymeric composite composition or the biodegradable polymer and the at least one additive. This step may be implemented thanks to a hopper. As disclosed preferably an additive content may be between 10 and 30%. The extrusion allows also to mix the biodegradable polymer with the at least one additive.

The method according to the invention may comprise a step of cooling 130. The step of cooling may be implemented by water and/or air cooling. The step of cooling allows to cool the polymeric composite composition at the end of the extrusion and preferably at the end of the extruder. The cooling step may allow to obtain at least one filament. Preferably, the step of cooling allows to reach a temperature of around room temperature, for example between 15 and 20° C. According to an embodiment the speed of traction of the cooling step may be adjusted to obtain a predetermined diameter. For example, the diameter may be between 1,75 mm and 2,85 mm. This step may comprise a control step to verify the diameter of the filament.

The method may comprise a step of drying 140. The temperature may be between 65° C. and 105° C. The step of drying allows to dry the filament.

The method may comprise a step of design manufacturing 150.

Preferably the step of design manufacturing may comprise a step of 3D printing. This step may be implemented by a 3D printer. Preferably, the diameter of the nozzle is predetermined. The step of design manufacturing may comprise a step of determining the machine parameter. For example, the filing rate, dimension, layer height, roughness etc. in order to obtain a biodegradable carrier, preferably a biodegradable carrier according to the invention.

According to another embodiment, the design manufacturing step 150 may comprise an injection. This step may be implemented thanks to at least one mold. The mold may correspond to two half-shells. Preferably the mold is texturized. The temperature during the injection may be between 180 to 250° C. According to an embodiment the temperature of the mold may be around 30 to 50° C. Preferably the temperature remains constant during the process of injection. The injection may comprise a step of cooling. This step may be implemented thanks to water and/or air cooling in order to solidify the polymeric composite composition in the mold. The mold may be opened, or the two half-shells may be removed. In the embodiment with two half-shells, the two half-solidified polymeric composite composition may be welded preferably by ultrasound in order to form a biodegradable carrier.

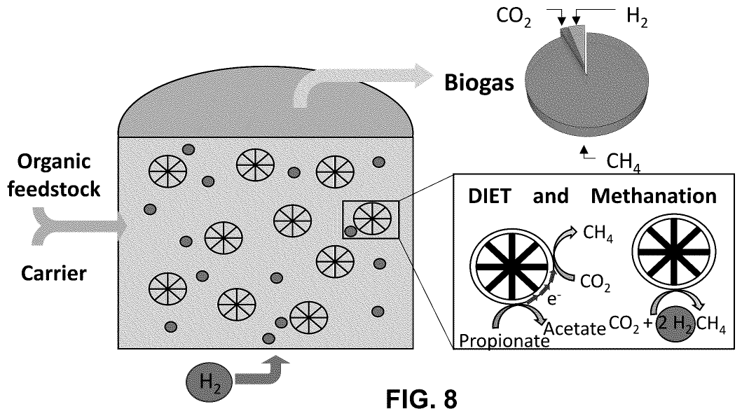

According to another aspect, the invention relates to a system suitable for methanization and/or methanation reaction, preferably for methanization and/or methanation reaction, comprising at least one methanization reactor and at least one biodegradable carrier for methanization and/or methanation reaction according to the invention. A system may be illustrated with the FIG. 8.

A system according to the invention allows to improve the production of biogas and its enrichment in biomethane. The system allows with the biodegradable carrier preferably according to the invention to promote the DIET and the methanation while reducing the supply of H₂. Indeed, in the reaction, the amounts of reactants involved in a methanization and/or methanation reaction are consumed to promote the presence of biomethane while allowing the proper development of microorganisms present in the system. The system allows to improve the control of reactions and the environment of microorganisms.

The number or the quantity of the biodegradable carrier in the methanization reactor may be according to the typology of the reactor. For example, the methanization reactor may comprise between 1% and 30% v/v (for volume to volume).

The invention can be the subject of numerous variants and applications other than those described above. In particular, unless otherwise indicated, the different structural and functional characteristics of each of the implementations described above should not be considered as combined and/or closely and/or inextricably linked to each other, but on the contrary as simple juxtapositions. In addition, the structural and/or functional characteristics of the various embodiments described above may be the subject in whole or in part of any different juxtaposition or any different combination.

1. A biodegradable carrier suitable for use in a methanization and/or methanation reaction comprising a methanization reactor, said biodegradable carrier comprising:
at least 70% by weight of a biodegradable polymer,
a closed cavity formed in the biodegradable polymer, and
a surface for growth of microorganisms,
wherein the biodegradable carrier is further configured to be in suspension in the methanization reactor.

2. The biodegradable carrier according to claim 1, wherein the biodegradable carrier is configured to be both floating and immersed in the methanization reactor.

3. The biodegradable carrier according to claim 1, wherein the methanization reactor is a biological in-situ methanation reactor.

4. The biodegradable carrier according to claim 1, wherein the biodegradable polymer comprises a polymeric composite composition.

5. The biodegradable carrier according to claim 1, wherein the biodegradable polymer comprises natural polymers, polysaccharides and/or proteins, polymers synthesized by bacteria, polyhydroxyalkanoate (PHA) and/or polyhydroxybutyrate (PHB) and/or polyhydroxyvalerates (PHV); and/or polyhydroxyhexanoate (PHH) and/or synthetic polymers derived from biotechnology of natural monomers, such as polylactic acid (PLA) and/or other aliphatic and/or aromatic polyesters and/or copolyesters, and/or mixture thereof.

6. The biodegradable carrier according to claim 1, wherein the biodegradable polymer forms the growth surface for micro-organisms.

7. The biodegradable carrier according to claim 1, wherein the biodegradable carrier presents a density between 0.70 and 1.00.

8. The biodegradable carrier according to claim 1, wherein the biodegradable carrier presents a conductivity between 10⁻² and 10² S/cm.

9. The biodegradable carrier according to claim 4, wherein the polymeric composite composition comprises at least one additive.

10. The biodegradable carrier according to claim 4, wherein the polymeric composite composition comprises at least one additive selected from: nanotube, biochar, carbon black, activated charcoal, glycerol, fiber, mineral additive and/or biological additive and/or mixture thereof.

11. The biodegradable carrier according to claim 4, wherein the polymeric composite composition comprises an additive content between 10% et 30% by weight.

12. The biodegradable carrier according to claim 1, wherein the closed cavity forms a total hollow volume of at least 10% of a volume of the biodegradable carrier.

13. The biodegradable carrier according to claim 1, wherein the biodegradable carrier presents at an indent and/or a hole and/or a segment.

14. The biodegradable carrier according to claim 1, wherein the biodegradable carrier has a biodegradable time between 60 and 120 days.

15. The biodegradable carrier according to claim 1, wherein the biodegradable carrier has a ratio of surface-volume between 200 and 1000m²/m³.

16. The biodegradable carrier according to claim 1, wherein the biodegradable carrier has a roughness having a parameter Ra between 100 and 200 μm.

17. Method for manufacturing a biodegradable carrier suitable for use in a methanization and/or methanation reaction comprising a methanization reactor, said method comprising:
A step of providing a polymeric composite composition,
A step of design manufacturing.

18. The method for manufacturing a biodegradable carrier suitable for use in a methanization and/or methanation reaction according to claim 17, further comprising a step of extruding.

19. The method for manufacturing a biodegradable carrier suitable for use in a methanization and/or methanation reaction according to claim 17, wherein the step of design manufacturing is selected between 3D printing such as stereolithography, laser sintering, multi-jet printing, modeling by fused deposition modelling and/or injection.

20. System for methanization and/or methanation reaction comprising a methanization reactor and he biodegradable carrier according to claim 1.
